(19) United States
(12) Patent Application Publication
(10) Pub. No.: US 2023/0357128 A1
(43) Pub. Date: Nov. 9, 2023

(54) UV-ABSORBING DONOR SPECIES FOR HIGH VISIBLE TRANSMITTANCE PHOTOVOLTAIC DEVICES

(71) Applicant: The Trustees of Princeton University, Princeton, NJ (US)

(72) Inventors: Melissa Ball, Princeton, NJ (US); Quinn Burlingame, Princeton, NJ (US); Lynn Loo, Princeton, NJ (US)

(21) Appl. No.: 18/013,610

(22) PCT Filed: Jun. 30, 2021
(86) PCT No.: PCT/US2021/039896

§ 371 Date: Dec. 29, 2022

Related U.S. Application Data
(60) Provisional application No. 63/046,315, filed on Jun. 30, 2020.

(51) Int. Cl. C07C 211/61; H01L 31/04

(52) U.S. Cl. CPC C07C 211/61; H01L 31/04; C07C 2603/18

(57) ABSTRACT

Near-ultraviolet (NUV) absorbing compounds are described herein which, in some embodiments, provide enhanced optoelectronic properties and visible light transmittances when employed as organic electron donors in various photovoltaic architectures. Photovoltaic devices incorporating such NUV-absorbing compounds in the active layer are also described.

### RELATED APPLICATION DATA

The present application claims priority pursuant to 35 U.S.C. § 119(e) to U.S. Provisional Patent Application Ser. No. 63/046,315 filed Jun. 30, 2020 which is incorporated herein by reference in its entirety.

### STATEMENT OF GOVERNMENT RIGHTS

This invention was made with government support under Grant Nos. 1843743 and 1420541 awarded by the National Science Foundation. The government has certain rights in the invention.

### FIELD

The present invention relates to compounds having ultra-violet (UV) absorption spectra and, in particular, to use of such compounds in the production of photovoltaic devices having high visible transmittance.

### BACKGROUND

Transparent photovoltaics (TPVs) have recently garnered significant research attention from the organic photovoltaic (OPV) and perovskite solar cell (PSC) communities due to the excellent match between TPV's application requirements and the tunable optical properties of the photoactive materials that compose these devices. Most commonly, TPVs maintain partial transparency by employing absorbers that selectively harvest the photon-rich near-infrared (NIR) portion of the solar spectrum to produce large photocurrents and high power-conversion efficiencies (PCEs). While the device structure, active layer thickness, and absorption profile of the active materials in such solar cells can be designed to optimize tradeoffs between power output and aesthetics, parasitic absorption of low energy visible light by NIR absorbers intrinsically limits their optical properties.

In contrast, solar cells with absorbers that target near-ultraviolet (NUV) wavelengths can avoid this parasitic visible light absorption to maximize transparency and color neutrality, albeit at the expense of power generation, making them an attractive option for driving low-power window-integrated electronics, such as electrically-dimmable smart windows, heads-up-displays, remote sensors, and internet-of-things devices.

While several transparent NUV-PSCs have been demonstrated, transparent NUV-OPVs have not been reported despite several recent examples of OPVs with NUV-absorbing active layers and opaque metal electrodes. This shortcoming is in part due to the lack of NUV-absorbing organic materials that meet the stringent optical and electronic requirements for integration into TPVs. For example, in the absence of a reflecting metal top electrode, optical simulations show that thick active layers (>100 nm) with high absorption coefficients (>2×10⁵ cm⁻¹) are needed to achieve near-unity optical absorption. However, maintaining efficient charge extraction with such thick active layers requires the absorbers to simultaneously exhibit high charge carrier mobilities and form heterojunctions (HJs) with excellent charge-transfer state dissociation efficiencies, so non-geminate recombination is minimized.

### SUMMARY

In view of these disadvantages, NUV-absorbing compounds are described herein which, in some embodiments, provide enhanced optoelectronic properties when employed as organic electron donors in various photovoltaic architectures. In some embodiments, a compound is of Formula (I):

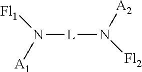

wherein A₁ and A₂ are independently selected from the group consisting of aryl and heteroaryl; and
wherein Fl₁ and Fl₂ are independently selected form the group consisting of fluorenyl and substituted fluorenyl; and
wherein L is selected from the group consisting of a fused aromatic ring structure and oligoarylene, wherein the oligoarylene comprises at least three arylene or heteroarylene units. As described further herein, compounds of Formula (I) can exhibit peak absorption in the NUV, while maintaining minimal absorbance in the visible region of the electromagnetic spectrum.

In another aspect, organic photovoltaic devices are described herein. An organic photovoltaic device, in some embodiments, comprises an anode, a cathode, and at least one active layer residing between the anode and the cathode, the active layer comprising an organic electron donor and organic electron acceptor, the organic electron donor comprising a compound of Formula (I):

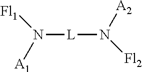

wherein A₁ and A₂ are independently selected from the group consisting of aryl and heteroaryl; and
wherein Fl₁ and Fl₂ are independently selected form the group consisting of fluorenyl and substituted fluorenyl; and
wherein L is selected from the group consisting of a fused aromatic ring structure and oligoarylene, wherein the oligoarylene comprises at least three arylene or heteroarylene units.

Peak absorbance of the photovoltaic can be in the range of 250 nm to 440 nm with minimal absorbance in the visible region. In some embodiments, for example, photovoltaic devices described here can have an average photopic-response-weighted visible transmittance of at least 75% or at least 80%. Moreover, the photovoltaic devices may also provide a color rendering index of at least 90.0%.

Such absorbance and transmittance profiles render the photovoltaic device suitable for application in several unique areas, including electrochromic devices. The photovoltaic devices, for example, can be vertically integrated with an electrochromic assembly. In some embodiments, the photovoltaic device can be vertically integrated with the electrochromic assembly over the same areal footprint. Alternatively, the photovoltaic device is spatially separate from the electrochromic assembly.

Photovoltaic devices described herein can also find application in UV filtering and/or harvesting. In some embodiments, the UV-absorbing photovoltaic devices can replace existing UV protector coatings of silicon and/or other photovoltaic technologies. By absorbing in the UV portion of the solar irradiation, photovoltaic devices described here can act as the UV protector for the silicon and/or other photovoltaic technologies, including organic active layer photovoltaics sensitive to UV degradation, while transmitting the visible and infrared light for power generation by the silicon and/or other photovoltaic devices. Vertically-integrated UV-absorbing photovoltaics described here can also be connected with the silicon and/or other photovoltaic devices externally in order to harvest the otherwise unused UV light for power generation.

The photovoltaic devices can also find application in energy storage architectures. In some embodiments, a battery architecture comprises an electrochemical assembly and a NUV-absorbing photovoltaic device described herein in electrical communication with the electrochemical assembly.

In another aspect, an organic photovoltaic device comprises an anode, a cathode, and at least one active layer residing between the anode and the cathode, the active layer comprising an organic electron donor and organic electron acceptor, the organic electron donor comprising diamine moieties linked by a fused aromatic ring structure or oligoarylene, wherein the organic photovoltaic device has an average photopic-response-weighted visible transmittance of at least 75% or at least 80%. In some embodiments, the electron donor of the active layer is a compound of Formula (I) described herein.

In a further aspect, methods of tuning UV-absorption of organic electron donors is described. In some embodiments, a method comprises tuning UV-absorption of an organic electron donor via varying the length of an oligoarylene or fused aromatic ring structure bridging two diamine moieties. In some embodiments, for example, the length of the oligoarylene bridge or fused aromatic ring bridge is varied to provide the organic electron donor a difference between the highest occupied molecular orbital (HOMO) and the lowest unoccupied molecular orbital (LUMO) of at least 2.8 eV or at least 3 eV.

These and other embodiments are further described in the following detailed description.

### BRIEF DESCRIPTION OF THE DRAWINGS

FIG. 1 illustrates altering the length of the oligoarylene bridge or fused aromatic ring bridge and the resultant effect on HOMO/LUMO energy levels of compounds for Formula (I) according to some embodiments.

FIG. 2 illustrates chemical structures of five donor molecules of Formula (I) according to some embodiments described herein.

FIG. 3A illustrates energy levels of electron donor species according to some embodiments described herein.

FIG. 3B illustrates HOMO and LUMO of BF-DPN generated by DFT.

FIG. 3C provides thin-film absorption coefficients for five electron donors and one electron acceptor according to some embodiments described herein.

FIGS. 4A and 4B illustrate J-V characteristics and EQE spectra, respectively, of opaque OPVs employing the five organic electron donors with the conventional device structure shown in FIG. 4C, according to some embodiments.

FIG. 4C illustrates convention and inverted photovoltaic architectures employing electron donor species described herein, according to some embodiments.

FIGS. 4D and 4E illustrate J-V characteristics and EQE spectra, respectively, of transparent photovoltaic devices comprising donors BF-DPB, BF-DPT, and BF-DPN in the inverted device structure shown in FIG. 4C.

FIG. 4F illustrates transmittance spectra of full-stack transparent OPVs described herein with the photopic luminosity function, V(λ), shown for reference, according to some embodiments.

FIGS. 5A-5C illustrate PCE vs. APT, PCE vs. CRI, and CIE x and y chromaticity coordinates, respectively, for transparent OPVs comprising BF-DPB, BF-DPT, and BF-DPN, according to some embodiments.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, oxygen and/or sulfur.

The term “fluorenyl” as used herein, alone or in combination, refers to a structure of the formula:

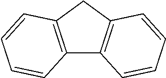 [SMILES: c1ccc2c(c1)Cc1ccccc1-2]

### I. UV-Absorbing Compounds

In one aspect, NUV-absorbing compounds are described herein. The compound can find application in photovoltaic devices as photoactive electron donor materials. In some embodiments, a compound is of Formula (I):

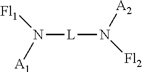

wherein A₁ and A₂ are independently selected from the group consisting of aryl and heteroaryl; and
wherein Fl₁ and Fl₂ are independently selected form the group consisting of fluorenyl and substituted fluorenyl; and
wherein L is selected from the group consisting of a fused aromatic ring structure and oligoarylene, wherein the oligoarylene comprises at least three arylene or heteroarylene units. As described further herein, compounds of Formula (I) can exhibit peak absorption in the NUV, while maintaining minimal absorbance in the visible region of the electromagnetic spectrum.

In some embodiments, A₁ and A₂ are the same aryl or heteroaryl group. Alternatively, A₁ and A₂ are different. Moreover, Fl₁ and Fl₂ can be the same or different. In some embodiments, at least one of Fl₁ and Fl₂ is substituted with one or more alkyl or alkenyl substituents. Substitution can occur at any desired position on the fluorenyl structure. For example, in some embodiments, at least one of Fl₁ and Fl₂ is of the formula:

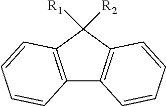

wherein R₁ and R₂ are independently selected from the group consisting of alkyl and alkenyl.

As described further herein, the fused aromatic ring structure or the oligoarylene can be employed to tune or alter the UV-absorption characteristics of compounds of Formula (I). In some embodiments, the fused aromatic ring structure is of the formula:

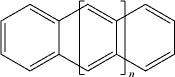

wherein n is an integer from 0 to 5. When n=0, for example, the fused aromatic structure is naphthalene, and when n=1, the fused aromatic structure is anthracene. In some embodiments, the fused aromatic ring structure comprises one or more heteroarylene units. In some embodiments, heteroarylene units comprise thiophene, pyrrole, azole, or combinations thereof. The fused aromatic ring structure can consist solely of the heteroarylene units or also include arylene units in a repeating or non-repeating arrangement. Arylene and/or heteroarylene units of the fused aromatic ring structure can have the same number of ring atomic positions or differing numbers of ring atomic positions. In some embodiments, for example, the fused aromatic ring structure comprises a mixture of phenylene and thiophene units.

The moiety or structure L bridging the two diamine moieties can also be an oligoarylene comprising at least three arylene or heteroarylene units. The oligoarylene, for example, can have the general structure:

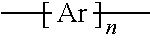

wherein Ar is arylene, heteroarylene, or various combinations thereof, and n is an integer from 3 to 10. In some embodiments described herein, n may also be 1 or 2. Varying the length of the oligoarylene linker or bridge can affect HOMO-LUMO offsets of the compound, as detailed herein.

Compounds of Formula (I), in some embodiments, exhibit a difference between the highest occupied molecular orbital (HOMO) and the lowest unoccupied molecular orbital (LUMO) of at least 2.5 eV, 2.8 eV, or at least 3 eV. In some embodiments, the HOMO-LUMO difference is at least 3 eV to 4 eV. Correspondingly, compounds of Formula (I) can exhibit peak absorption in a range 250 nm to 440 nm, in some embodiments. Peak absorption of compounds of Formula (I) may also lie in the range for 300 nm to 410 nm, depending of compound structure and specific identity of the L group bridging the diamine moieties.

### II. Organic Photovoltaic Devices

In another aspect, organic photovoltaic devices are described herein. An organic photovoltaic device, in some embodiments, comprises an anode, a cathode, and at least one active layer residing between the anode and the cathode, the active layer comprising an organic electron donor and organic electron acceptor, the organic electron donor comprising a compound of Formula (I):

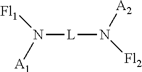

wherein A₁ and A₂ are independently selected from the group consisting of aryl and heteroaryl; and
wherein Fl₁ and Fl₂ are independently selected form the group consisting of fluorenyl and substituted fluorenyl; and
wherein L is selected from the group consisting of a fused aromatic ring structure and oligoarylene, wherein the oligoarylene comprises at least three arylene or heteroarylene units. Compounds of Formula (I) employed in active layers of photovoltaic devices described herein can have any structure and/or properties provided in Section I above.

Organic electron donor and organic electron acceptor of the active layer can exhibit any electromagnetic radiation absorption profile not inconsistent with the objectives of the present invention. In some embodiments, organic electron donor and acceptor exhibit peak absorbance in the range of 250 nm to 440 nm or 300 nm to 410 nm. In such embodiments, the active layer is largely transparent to light in the visible and near infra-red regions. For example, the active layer can generally display an average transmittance in the visible light region of 60 percent to 100 percent. Average visible light transmittance of an active layer described herein can also have a value selected from the following table.

```
Average
Visible Light
and/or Infrared
Transmittance
of Active Layer
(%)
  70-100
  75-100
  80-100
70-98
80-98
80-95
85-98
```

As discussed further herein, active layers having the foregoing transmittance values render the photovoltaic devices particularly suited for applications where transparency in the visible light region and/or infrared light region is a key requirement, such as windows for commercial and industrial buildings, homes and transportation vehicles including cars, buses, trucks, trains and airplanes. The foregoing transmittance values also contribute to the overall photovoltaic construction achieving high values for average photopic-response-weighted transmittance discussed further herein.

In addition to various organic electron donor and acceptor species, the active layer can have any architecture not inconsistent with the objectives of the present invention. In some embodiments, a planar heterojunction is formed between adjacent layers of organic electron donor and organic electron acceptor. In such embodiments, thickness of the organic electron donor layer and organic electron acceptor layer can be selected according to several considerations including sufficient light absorption by the active layer and exciton diffusion path lengths. Organic electron donor and acceptor layers can generally have individual thicknesses of 10 to 400 nm. In some embodiments, individual thicknesses of organic electron donor and acceptor layers can be 20 nm to 300 nm.

In some embodiments, the active layer exhibits a gradient heterojunction architecture. In such an architecture, organic electron donor gradually decreases from 100 percent at the anode side to zero percent at the cathode side of the active layer. Similarly, organic electron acceptor gradually decreases from 100 percent from the cathode side to zero percent at the anode side of the active layer.

In other embodiments, the active layer can exhibit a mixed heterojunction or bulk heterojunction architecture wherein organic electron donor and acceptor are mixed or dispersed throughout one another. In some embodiments, organic electron donor and organic electron acceptor are mixed in solution and deposited to form the active layer. The active layer is subsequently annealed to induce spinodal decomposition or phase separation of the active layer, thereby forming mixed heterojunction architectures. In some embodiments, the organic donor and organic electron acceptor are co-deposited from separate solutions or from the gas-phase to form a mixed active layer. In further embodiments, the active layer can have any combination of planar heterojunction, gradient heterojunction and/or mixed heterojunction architectures. Additionally, the active layer can be pinhole-free, improving device areal scalability and fabrication yield. Organic electron donor and organic electron acceptor can be mixed at any desired ratio to provide the bulk heterojunction. Mixing ratio can be dependent upon several considerations including, but not limited to, specific chemical identities of the donor and acceptor materials, desired absorption/transmittance and charge transfer properties of the active layer, and overall performance of the photovoltaic device. In some embodiments, ratio of the organic donor to the organic electron acceptor ranges from 1:10 to 10:1. Additionally, bulk heterojunction active layers can have any desired thickness. In some embodiments, a bulk heterjunction active layer has thickness of 10 nm to 500 nm, 20 nm to 300 nm, or 50 nm to 300 nm. Thickness of the bulk heterojunction layer can be varied according to specific chemical identities of the organic electron donor and acceptor and the desired electronic and visible transmittance properties of the photovoltaic architecture.

In addition to the active layer, photovoltaic devices described herein can comprise one or more charge transport layers and/or exciton blocking layers. A hole transport layer (HTL) or exciton blocking layer, in some embodiments, is positioned between the anode and active layer. For example, an HTL can comprise one or more organic or inorganic layers such as poly(3,4-ethylenedioxythiophene):poly(styrenesulfonate) PEDOT:PSS, polyaniline-poly(2-acrylamido-2-methyl-1-propane-sulfonic acid) PANI-PAAMPSA and/or transition metal oxide. Suitable transition metal oxide can comprise molybdenum oxide, MoOx, where x denotes any ratio of Mo to O. Additional transition metal oxides include vanadium oxide, nickel oxide or oxides of similar electronic structure.

An electron transport layer (ETL) or exciton blocking layer, in some embodiments, is positioned between the cathode and the active layer. An ETL can comprise one or more organic or inorganic layers such as bathocuproine (BCP), calcium fluoride, lithium fluoride, bathophenanthroline doped with lithium (Li:BPhen), poly[(9,9-bis(3-(N,N dimethylamino)propyl)-2,7-fluorene)-alt-2,7-(9,9-dioctylfluorene)] PFN, 2,2′,2″-(1,3,5-benzinetriyl)-tris(1-phenyl-1-H-benzimidazole) (TPBi) and/or transition metal oxide. Suitable transition metal oxide for an ETL can comprise titanium oxide (TiOₓ) and/or zinc oxide. HTL and/or ETL layers can be deposited by any method not inconsistent with the objectives of the present invention. HTL and/or ETL layers can be deposited by sputtering or thermal evaporation. In other embodiments, HTL and/or ETL layers can be deposited by solution-based techniques such as spin coating, blade coating, knife coating, slot-die coating, screen printing, flexographic printing, Gravure printing, ink jet printing or spray coating. In further embodiments, HTL and/or ETL layers may be deposited by lamination.

The anode and/or cathode of a photovoltaic device described herein, in some embodiments, is formed of a radiation transmissive material. In being radiation transmissive, the anode and/or cathode is transparent or substantially transparent for portions of the electromagnetic spectrum characteristic of solar spectral irradiation. In some embodiments, the anode and/or cathode are formed of a radiation transmissive metal oxide. Suitable radiation transmissive metal oxides can include tin oxide (ITO), gallium indium tin oxide (GITO), zinc indium tin oxide (ZITO), indium zinc oxide (IZO) and silver zinc oxide (AZO) and chemically-functionalized versions of metal oxides such as fluorine-doped tin oxide (FTO). In other embodiments, radiation transmissive materials for the anode and/or cathode can include organic materials such conductive or semiconductive polymeric species. Suitable polymeric species can comprise polyaniline (PANI) and its chemical relatives, such as PANI-PAAMPSA. In some embodiments, 3,4-polyethylenedioxythiophene (PEDOT) can be a suitable radiation transmissive polymeric material for the anode and/or cathode. Nanowire constructions can also be used as radiation transmissive material for the anode and/or cathode. In some embodiments, for example, a radiation transmissive anode and/or cathode can be a metal-nanowire mesh, such as silver nanowires dispersed in polymeric matrix. Metal films of sufficient thinness to transmit significant near-ultraviolet, visible and/or infrared radiation may also be employed as anode and/or cathode. In some embodiments, the anode and/or cathode exhibit average transmittance in the visible light region of 80 percent to 100 percent. In other embodiments, the anode or cathode may be opaque. The anode or cathode for example, may be formed of metal, such as aluminum, silver or copper of sufficient thickness to reflect light or otherwise block light transmission. Moreover, thin-film layers of molybdenum oxide or LiF can be employed adjacent to, or mixed with, transmissive or opaque anode and/or cathode architectures to improve photovoltaic device performance or device transparency in the visible and/or infrared region(s).

In some embodiments, a light outcoupling layer positioned over the anode or cathode. A light outcoupling layer can assist with enhancing transmittance of visible and/or infrared radiation through the photovoltaic architecture. The outcoupling layer can comprise any composition enhancing extraction of visible light from or passage of visible light through the photovoltaic architectures, including anode, cathode, active layer and any exciton transport layers. In some embodiments, for example, an outcoupling layer comprises TPBi. In such embodiments, the TPBi outcoupling layer can be applied to a radiation transmissive anode, wherein the photovoltaic device adopts an inverted architecture. In addition to inverted architectures, photovoltaic devices described herein can adopt conventional architectures. The outcoupling layer can have any desired thickness consistent with enhancing transmission/passage of visible and/or infrared radiation through the photovoltaic architecture. In some embodiments, an outcoupling layer has a thickness of 10 nm to 500 nm or 40 nm to 120 nm. Photovoltaic devices employing compounds of Formula (I) described herein can exhibit a single-junction architecture or multi-junction architecture.

Photovoltaic devices having composition and architectures described herein, in some embodiments have an open circuit voltage (V_{oc}) of at least 1.5 V or at least 1.7 V. In some embodiments, the photovoltaic devices have a V_{oc} of at least 1.5 V to 3 V. Moreover, the photovoltaic devices, in some embodiments, exhibit an average photopic-response-weighted visible transmittance of at least 75% or at least 80%. A photovoltaic device, for example, can have an average photopic-response-weighted visible transmittance of 80% to 85%. Additionally, photovoltaic devices described herein can provide a color rendering index (CRI) of at least 90.0 or at least 95.0. In some embodiments, a photovoltaic device provides a CRI of 95.0 to 98.0.

In another aspect, a photovoltaic device described herein comprises an anode, a cathode, and at least one active layer residing between the anode and the cathode, the active layer comprising an organic electron donor and organic electron acceptor, the organic electron donor comprising diamine moieties linked by a fused aromatic ring structure or oligoarylene, wherein the organic photovoltaic device has an average photopic-response-weighted visible transmittance of at least 75%. Such a photovoltaic device can have any construction, composition, and/or properties described above for a photovoltaic device in this Section II. In some embodiments, for example, the organic electron donor comprises a compound of Formula (I):

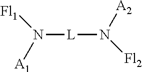

wherein A₁ and A₂ are independently selected from the group consisting of aryl and heteroaryl; and
wherein Fl₁ and Fl₂ are independently selected form the group consisting of fluorenyl and substituted fluorenyl; and
wherein L is selected from the group consisting of the fused aromatic ring structure and the oligoarylene, wherein the oligoarylene comprises at least two arylene or heteroarylene units.

### III. Electrochromic and Other Devices

In addition to modulating the transmission of visible light into buildings and transportation vehicles in order to augment lighting/shading needs and/or to provide privacy, solar-powered electrochromic devices described herein can also modulate the transmission of infrared light into buildings and transportation vehicles in order to augment heating/cooling needs. In some embodiments, an electrochromic device comprises an electrochromic assembly and a photovoltaic device in electrical communication with the electrochromic assembly for switching the electrochromic assembly between light and dark states via application of a photovoltage, wherein the photovoltaic device has composition, construction and/or properties described in Section II above.

Moreover, the electrochromic assembly comprises a single electrochromic layer. In some embodiments, the electrochromic assembly comprises a plurality of electrochromic layers. The one or more electrochromic layers of the assembly can exhibit redox properties switchable between light/clear and dark/opaque states by photovoltages provided by photovoltaic devices described herein.

As described herein, the photovoltaic devices can be transparent in the visible and/or infrared and thus be vertically integrated with the electrochromic assembly. In some embodiments, the photovoltaic device is vertically integrated over the same areal footprint as the electrochromic assembly. Vertical integration of the single-junction photovoltaic device with the electrochromic assembly can greatly simplify application of electrochromic devices to a variety of products including windows for commercial and industrial buildings, homes and transportation vehicles including cars, buses, trucks, trains and airplanes. Electrochromic devices, for example, can be positioned between window panes for environmental isolation. Vertical integration of the photovoltaic apparatus can render the electrochromic device standalone, thereby greatly simplifying electrical wiring that complicates prior electrochromic apparatus installation. Alternatively, a single-junction photovoltaic device described herein is spatially separate or external to the electrochromic assembly.

In addition to electrochromic devices, photovoltaic devices having composition, architecture, and/or properties described herein can find use in applications where optical clarity in the visible region is advantageous or desirable including, but not limited to, heads-up-displays, remote sensors, and internet-of-things devices.

### IV. Methods of Tuning UV-Absorption

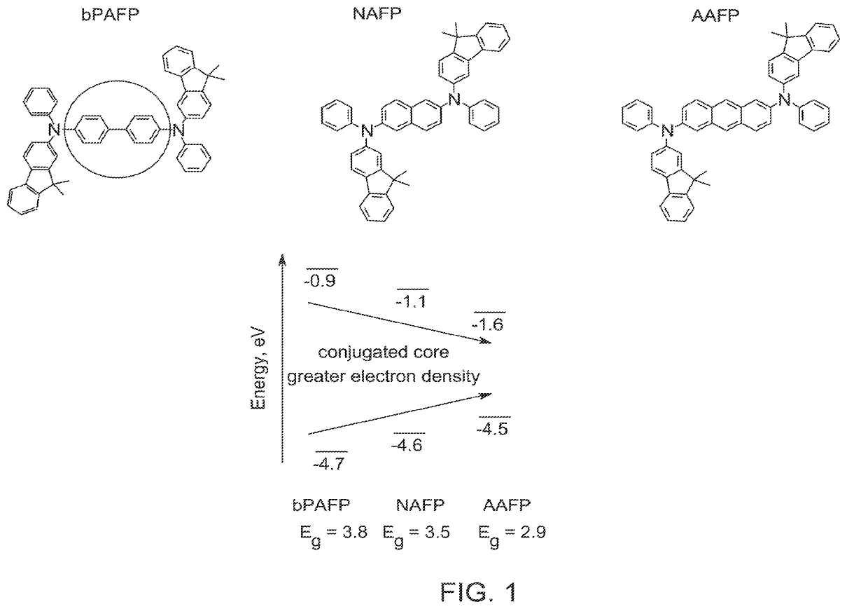

In a further aspect, methods of tuning UV-absorption of organic electron donors is described. In some embodiments, a method comprises tuning UV-absorption of an organic electron donor via varying length of an oligoarylene or fused aromatic ring structure bridging two diamine moieties. In some embodiments, for example, the length of the oligoarylene bridge or fused aromatic ring bridge is varied to provide the organic electron donor a difference between the highest occupied molecular orbital (HOMO) and the lowest unoccupied molecular orbital (LUMO) of at least 2.8 eV or at least 3 eV. FIG. 1 illustrates altering the length of the oligoarylene bridge or fused aromatic ring bridge and the resultant effect on HOMO/LUMO energy levels of compounds for Formula (I) according to some embodiments.

These and other embodiments are further illustrated by the following non-limiting examples.

### Example 1—Compounds of Formula (I) and Associated Photovoltaic Devices

In the present example, compounds of Formula (I) are synthesized and employed in photovoltaic devices having enhanced transmittance of visible electromagnetic radiation through the complete photovoltaic architecture. In particular, N,N-diaryl-diamine NUV-absorbing donor molecules with targeted absorption profiles and frontier orbital energies were synthesized to pair with B4PymPm to fabricate NUV-OPVs that maximize device efficiency without compromising transparency or color neutrality. The diamine moieties are electronically coupled via a molecular linker comprised of either an oligophenylene or an acene to systematically decrease the lowest unoccupied molecular orbital (LUMO) energy and absorption onset energy, while maintaining their highest occupied molecular orbital (HOMO) energies to retain high V_{OC}'s in solar cells when these donors are paired with B4PymPm. Each of these donor absorbers exhibit a peak absorption coefficient ≥2.5×10⁵ cm⁻¹, and solar cells containing these donors generate large V_{OC}'s between 1.74 V and 2.03 V. NUV-OPVs with opaque top metal electrodes were first fabricated with each of these donors to evaluate their photovoltaic performance, and the three most promising candidates were then incorporated into fully transparent inverted solar cells with an indium tin oxide (ITO) top electrode and a TPBi (2,2′,2″-(1,3,5-Benzinetriyl)-tris(1-phenyl-1-H-benzimidazole) optical outcoupling layer. These transparent NUV-OPVs are color neutral with near-perfect color rendering indices (CRIs) of 95.0-97.1, and they exhibit record average photopic-response-weighted visible transmittances (APTs) of 80.3-82.0%, while producing output power densities of 4.3-7.0 W/m² under simulated AM1.5G solar illumination.

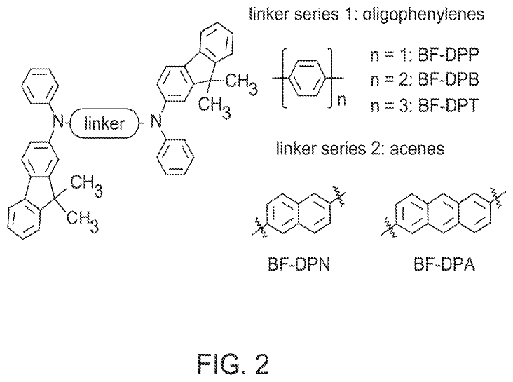

FIG. 2 illustrates chemical structures of five electron donor molecules of Formula (I) used in the present example. Each donor absorber can be deconstructed into two triaryl amines, which are symmetrically substituted with a phenyl and fluorene appendage, that are connected by either an oligophenylene or an acene molecular linker. These new donor absorbers are labeled as BF-DPL, where BF=bis-fluorene, DP=diphenyl, and L=linker. The number of phenylene units in the oligophenylene donors varies from n=1-3. Accordingly, these donors are BF-DPP, BF-DPB, and BF-DPT, where P=phenyl, B=biphenyl, and T=terphenyl, respectively. The donors in the acene linker series include BF-DPN and BF-DPA, where N=naphthalene and A=anthracene. A Buchwald-Hartwig coupling reaction was employed between 2-anilino-9,9-dimethylfluorene and the respective dibrominated linker to synthesize the new donor absorbers on gram scales and in high yields. All five donor absorbers have excellent thermal properties appropriate for thermal evaporation and sublimation purification without degradation, as evidenced by their high decomposition temperatures (T_{d}) from thermogravimetric analysis, tabulated in Table 1.

TABLE I
Material Properties of Absorbers

| | HOMO_{DFT}^{a} | HOMO_{UPS} | LUMO_{DFT}^{a} | LUMO_{IPES} | E_{g,DFT} | E_{G,exp.} | E_{G,opt} | λₒₙₛₑₜ | Tₘ^{b} | T_{sub}^{c} | T_{d}^{d} |
|---|---|---|---|---|---|---|---|---|---|---|---|
| Molecule | (eV) | (eV) | (eV) | (eV) | (eV) | (eV) | (eV) | (nm) | (° C.) | (° C.) | (° C.) |
| BF-DPP | −4.8 | −5.1 | −1.3 | −1.9 | 3.5 | 3.2 | 3.05 | 407 | 209 | 310 | 362 |
| BF-DPB | −4.9 | −5.2 | −1.3 | −1.8 | 3.6 | 3.4 | 3.00 | 414 | 267 | 330 | 397 |
| BF-DPT | −5.0 | −5.2 | −1.4 | −1.9 | 3.6 | 3.3 | 2.97 | 417 | 326 | 375 | 377 |
| BF-DPN | −4.9 | −5.2 | −1.5 | −2.1 | 3.4 | 3.1 | 2.83 | 438 | 270 | 345 | 383 |
| BF-DPA | −4.9 | −5.2 | −1.9 | −2.4 | 2.9 | 2.8 | 2.45 | 507 | 362 | 360 | 392 |
| B4PymPm | −7.1 | −7.2 | −2.9 | −2.9 | 4.2 | 4.3 | 3.46 | 358 | 377 | — | 402 |

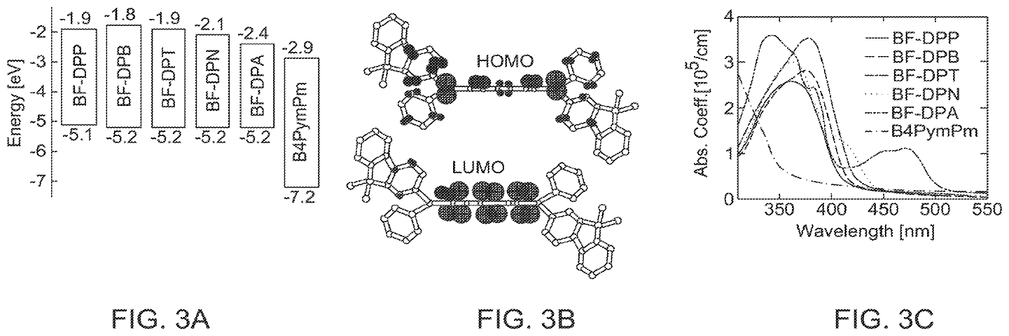

FIG. 3A contains the HOMO and LUMO energy levels from ultraviolet photoelectron spectroscopy (UPS) and inverse photoelectron spectroscopy (IPES), respectively. Using time-dependent density functional theory (TD-DFT), it was found that the donors' HOMOs comprise a pair of p-orbitals containing the two lone pair of electrons centered on the di-nitrogen atoms, as shown in FIG. 3B for BF-DPN. Each of the nitrogen atoms is bonded to three aryl groups, thus the HOMO energy is not strongly dependent on the linker. This is confirmed by the UPS measurements, which place the HOMO edge of all the donors at 5.1-5.2 eV below vacuum level, irrespective of the linker. In contrast, the LUMO shows more dependence on the choice of linker between the diamines. For donors in the acene series, the LUMO resides on a set of p-orbitals centered on the linker moiety, as shown in FIG. 2B for BF-DPN and as well as for BF-DPA (not shown). The stabilization of the LUMO, and thus the electron affinity of the material, increase with the number of fused rings. The LUMO becomes progressively centered on the linker as the number of phenylenes is increased from n=1 to n=3 in the oligophenylene series, however delocalization and stabilization of the LUMO are significantly smaller than with the acenes, consistent with the observed weaker dependence of the LUMO energy.

The absorption spectra for the five donor molecules and B4PymPm are presented in FIG. 3C. The trend in absorption onsets correlates with the size of the linker across the five donors; that is, the larger the linker, the smaller the optical gap. Each of the donors has a peak absorption coefficient ≥2.5×10⁵ cm⁻¹, and the absorption coefficient positively correlates with the size of the linker, as shown in FIG. 3C. With the help of TD-DFT, the lowest energy absorption feature in the absorption spectra of the acene-based donors was assigned to their HOMO→LUMO transitions. In the spectra of the donors in the oligophenylene series, the lowest energy absorption feature comprises several electronic transitions characterized by the promotion of an electron from a nitrogen-centered molecular orbital to the surrounding π-space. The frontier orbital energies and absorption spectra were calculated using TD-DFT, and the trend in the computationally- and experimentally-determined frontier orbital energies and absorption features are in good agreement, as tabulated in Table 1.

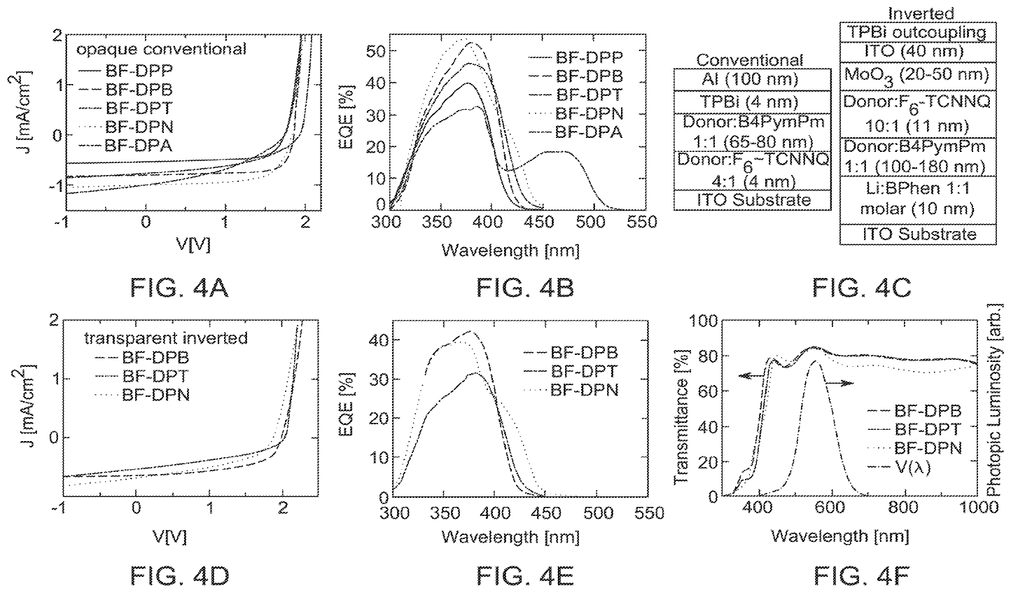

To test the photovoltaic performance of the donor absorbers, bulk-heterojunction (BHJ) OPVs were fabricated in the conventional structure with Al top electrodes, as shown in FIG. 4. The blend ratio of each HJ was optimized at a 1:1 donor: acceptor volume ratio, with optimized active layer thicknesses of 65 nm for BF-DPN, 70 nm for BF-DPT, and 80 nm for BF-DPP, BF-DPB, and BF-DPA HJs. Current density vs. voltage (J-V) characteristics of the solar cells under simulated AM1.5G solar illumination and external quantum efficiency (EQE) spectra for each optimized device are shown in FIGS. 4A and 4B, and their photovoltaic performance metrics, including J_{SC}, V_{OC}, fill factor (FF), and PCE, are compiled in Table 2. As expected, the EQE spectra mirror the absorption spectra of each donor, with little or no contribution from deep-UV absorbing B4PymPm. While the opaque (Al top metal electrode) NUV-OPVs with BF-DPP, BF-DPB, and BF-DPN extract charges efficiently based on the relatively flat J-V curves about V=0, the photocurrent in the opaque NUV-OPVs employing donors with the two largest aryl linkers, BF-DPT and BF-DPA is voltage-dependent, which limits their. FF's to 46% and 35%, respectively. The V_{OC}'s of all the OPVs tested cluster between 1.74 V and 1.93 V, consistent with the similarity of the HOMO energies across the donors.

TABLE 2
Photovoltaic and optical performance metrics of OPVs.

| | | HJ | J_{SC,EQE} | J_{SC,J-V} | | | PCE w/ | | | | |
|---|---|---|---|---|---|---|---|---|---|---|---|
| Device | Donor | Thickness | mA/ | mA/ | V_{OC} | FF | J_{SC,EQE} | APT | | CCT | |
| architecture | molecule | (nm) | cm² | cm² | (V) | (%) | (%) | (%) | CRI | (K) | CIE [x, y] |
| opaque, | BF-DPP | 80 | 0.56 | 0.54 | 1.74 | 66 | 0.64 | — | — | — | — |
| conventional | BF-DPB | 80 | 0.81 | 0.80 | 1.85 | 74 | 1.11 | — | — | — | — |
| | BF-DPT | 70 | 0.83 | 0.77 | 1.93 | 46 | 0.74 | — | — | — | — |
| | BF-DPN | 65 | 0.97 | 1.00 | 1.84 | 67 | 1.20 | — | — | — | — |
| | BF-DPA | 80 | 1.22 | 1.00 | 1.74 | 40 | 0.85 | — | — | — | — |
| opaque, | BF-DPB | 120 | 0.77 | 0.81 | 1.99 | 63 | 0.96 | — | — | — | — |
| inverted | BF-DPT | 100 | 0.62 | 0.60 | 2.03 | 38 | 0.46 | — | — | — | — |
| | BF-DPN | 180 | 0.98 | 0.86 | 1.88 | 42 | 0.77 | — | — | — | — |
| transparent, | BF-DPB | 120 | 0.64 | 0.64 | 2.00 | 55 | 0.70 | 81.8 | 97.1 | 5,312 | \|[0.337, 0.354] |
| inverted | BF-DPT | 100 | 0.53 | 0.53 | 2.03 | 40 | 0.43 | 82.0 | 96.6 | 5,242 | [0.339, 0.356] |
| | BF-DPN | 180 | 0.76 | 0.68 | 1.88 | 43 | 0.61 | 80.3 | 95.0 | 5,487 | [0.333, 0.354] |
| D. Liu et al. | — | — | — | 2.00 | 1.05 | 55 | 1.16 | 69.1 | 90.2 | 5,019^{†} | [0.348, |
| | | | | | | | | | | | 0.393]^{†} |
| Y. Li et al. | — | — | — | 16.3 | 0.73 | 68 | 8.1 | 43.3 | 86.0 | 4,143 | [0.38, 0.39] |

D. Liu, C. Yang, P. Chen, M. Bates, S. Han, P. Askeland, R. R. Lunt, ACS Appl. Energy Mater. 2019, 2, 3972.
Y. Li, X. Guo, Z. Peng, B. Qu, H. Yan, H. Ade, M. Zhang, S. R. Forrest, Proc. Natl. Acad. Sci. U.S.A. 2020, 1.

Based on the performance of the conventional NUV-OPVs described above, BF-DPA and BF-DPP were ruled out as candidates for transparent NUV-OPVs because of poor charge extraction in the former and weak absorption and low J_{SC} in the latter. To fabricate the transparent NUV-OPVs, the choice of top electrode and the optical outcoupling structure are important design considerations. Thus, in this example, sputtered ITO top electrodes were employed with TPBi outcoupling layers to realize the full potential for color neutrality and high transmittance of the fabricated solar cells. To yield transparent NUV-OPVs with 40-nm thick ITO top electrodes, we find that a MoO₃ layer is required to protect the organics during ITO sputtering. Since MoO₃ is a deep work-function hole transporting layer, its use atop the heterojunction required a switch from the conventional architecture to the inverted architecture shown in FIG. 4C. Inverted OPVs were additionally fabricated with Al electrodes to assess the impact of replacing Al top electrodes with ITO in the inverted architecture. Indium tin oxide deposition initially resulted in transparent NUV-OPVs that were partially shunted or, in some cases, fully short-circuited. Applying a large reverse bias for several seconds removed shunts and allowed the recovery of the performance of the diodes in most cases. The inverted NUV-OPVs are able to sustain remarkably high reverse biases of −20V, corresponding to an effective electric field of >1.2 MV/cm, which makes them particularly amenable to this de-shunting treatment.

The J-V characteristics and EQE spectra of the optimized transparent NUV-OPVs are shown in FIGS. 4D and 4E, respectively, and their photovoltaic performance metrics are tabulated in Table 2 along with those of inverted, opaque NUV-OPVs with Al anodes for comparison. In each case, the optimum active layer for the inverted NUV-OPVs is thicker than that of their opaque conventional counterparts to compensate for the lack of a reflecting electrode. In particular, the active layer of NUV-OPVs containing BF-DPN is 180 nm thick, compared to just 65 nm in its optimized opaque conventional counterpart to compensate for weak absorption of photons between 400 nm and 440 nm. Consistent with the performance of its conventional counterpart, charge extraction in the transparent NUV-OPVs containing BF-DPT is less efficient, even with a relatively thin active layer of 100 nm, leading to a FF of 40%. The BF-DPB-containing device is the highest performing transparent NUV-OPV with a FF of 55% at an active layer thickness of 120 nm. Its J_{SC} of 0.64 mA/cm² is just 20% less than that generated by both its opaque inverted and conventional counterparts, whose photocurrents benefit from reflecting metal top electrodes. Interestingly, all the inverted devices produce V_{OC}'s that are higher than their OPV counterparts constructed in the conventional architecture, presumably due to reduced charge build up and recombination at the Li:BPhen (bathophenanthroline) hole-transport layer and BF-DPL:F₆-TCNNQ electron-transport layer interfaces.

The transmittance spectrum of each full-stack, functional transparent NUV-OPV has peaks and valleys corresponding to optical interference minima and maxima. By growing an outcoupling layer above the ITO anode, the effective optical thickness of the transparent NUV-OPVs can be modulated to increase APT by shifting these interference fringes to better align with the photopic luminosity function of the eye (FIG. 4F, right-axis). For this purpose, TPBi outcoupling layer thicknesses was tailored on each transparent NUV-OPV (100 nm for BF-DPB, 90 nm for BF-DPT, and 50 nm for BF-DPN) to produce the transmittance spectra shown in FIG. 3F. An additional benefit of using TPBi as an outcoupling layer is that its refractive index lies between that of ITO and air, which reduces reflection across all wavelengths.

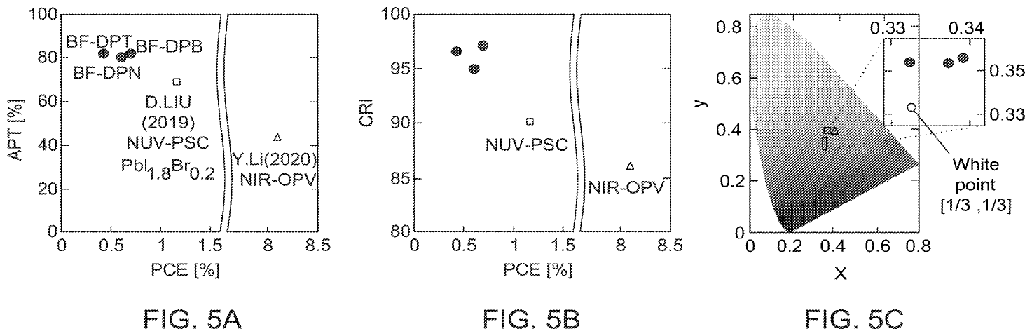

In addition to the APT of each device, the CRI, the correlated color temperature (CCT) and Commission Internationale de l'Éclairage (CIE) chromaticity coordinates of AM1.5G sunlight transmitted through each transparent NUV-OPV were calculated and tabulated the results in Table 2. At 97.1, the CRI of the BF-DPB transparent NUV-OPV is the highest reported for any transparent solar cell, and the APTs and CRIs of all three transparent NUV-OPVs presented here are the highest among OPVs, and are on par with luminescent solar concentrators, and the most transparent NUV-absorbing PSCs. It was also find that the CCT's are within 300 K of natural sunlight, which has a CCT of 5500 K, and that the CIE chromaticity coordinates for each device are within Δx=0.01 and Δy=0.03 of the white point at [⅓, ⅓], demonstrating the excellent color neutrality of the transparent NUV-OPVs. To put these values into context, we plotted PCE vs. APT, PCE vs. CRI, and chromaticity coordinates on the CIE 1931 color space for each transparent NUV-OPV in this work, as well as a top-performing transparent NUV-absorbing PSC and a NIR-absorbing TPV from the literature in FIGS. 5A-5C. The aesthetic benefits and performance tradeoffs of the present approach are evident from these plots.

In this non-limiting example, two series of NUV-absorbing donor molecules were designed with absorption profiles that have been systematically tuned by varying the aryl linker between their diamine moieties with either an oligophenylene or an acene. The best performing transparent NUV-OPVs presented here produce power densities of 6.1-7.0 W/m² under simulated AM1.5G illumination, and boast APTs of 80.3-81.8% and CRIs of 95.0-97.1, making these organic cells the most color neutral and transparent to-date. These results highlight the potential of transparent NUV-OPVs for applications that require low power and maximum optical clarity, for example, to power electrically-dimmable smart windows that value a large transmittance contrast between their clear and tinted states.

### Methods

### Synthesis and Characterization

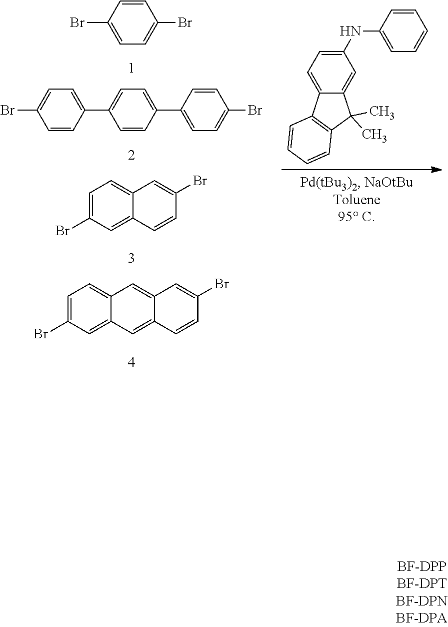 [SMILES: Brc1ccc(-c2ccc(-c3ccc(Br)cc3)cc2)cc1 ; Brc1ccc(Br)cc1 ; CC1(C)c2ccccc2-c2ccc(Nc3ccccc3)cc21 ; Brc1ccc2cc(Br)ccc2c1 ; Brc1ccc2cc3cc(Br)ccc3cc2c1]

### General Procedure:

The respective dibrominated linker (1-4) and 2-anilino-9,9-dimethylfluorene (1.00:2.01 molar equivalents) were added to a flame-dried round bottom flask along with sodium tert-butoxide (3 equivalents) and the reagents were cycled three times between nitrogen gas and vacuum. The reaction was left under nitrogen gas and anhydrous toluene was added to the flask (100 mg of dibrominated starting material=10.0 ml of toluene). The reaction was then degassed with a nitrogen inlet and an outlet needle for thirty minutes. Tri-tert-butylphosphine was added to the flask (0.100 mol percent) and the reaction was subsequently degassed for an additional fifteen minutes. The reaction was sealed and placed in an oil bath at 95° C. overnight. The reaction was cooled, filtered, and washed with copious amounts of diethyl ether. The solids were collected and subjected to sublimation for final purification. BF-DPP solids did not crash out of the reaction, and a column using a gradient to 50% DCM to 50% hexanes was ran. All reactions ran on gram scales except BF-DPA, which was ran at 1.5 mmol/0.500 gr scale.

BF-DPP Yellow solid, reaction yield=87% (2.38 gr), sublimation yield=34%. ¹HNMR (500 MHz, 295K, DMSO) δ 7.74 (d, 2H), 7.73 (d, 2H), 7.50 (d, J=7.4 Hz, 2H), 7.33-7.21 (br, 10H), 7.07 (d, J=8.0 Hz, 4H), 7.02-6.98 (br, 8H), 1.36 (s, 12H). ¹³CNMR (125 MHz, 295K, DMSO) δ 158.86, 157.20, 151.50, 150.92, 146.60, 142.39, 137.58, 133.61, 131.16, 130.69, 129.39, 127.21, 126.76, 126.74, 126.64, 125.19, 123.61, 122.00, 50.50, 30.92. HRMS (MALDI+) calculated m/z for [C₄₈H₄₀N₂+H]+ is 644.32, found 644.34.

BF-DPT Yellow solid, reaction yield=87% (2.19 gr), sublimation yield=27%. ¹HNMR (500 MHz, 295K, C₂D₄Cl₂) δ 7.67 (br s, 4H), 7.65 (d, J=7.5 Hz, 2H), 7.62 (d, J=8.1 Hz, 2H), 7.57 (d, J=8.6 Hz, 4H), 7.40 (d, J=7.4 Hz, 2H), 7.31-7.24 (br, 10H), 7.17 (dd, J=8.7, J=2.4 Hz, 8H), 7.07-7.04 (br, 4H), 1.42 (s, 12H). ¹³CNMR (125 MHz, 295K, C₂D₄Cl₂) δ 129.69, 129.17, 127.80, 127.39, 127.17, 127.00, 124.79, 124.14, 124.12, 123.38, 122.90, 121.04, 119.81, 119.53, 47.18, 27.27. *the carbon spectra was not able to capture protons between 160 and 130 ppm due to solubility. HRMS (MALDI+) calculated m/z for [C₆₀H₄₈N₂]+ is 796.38, found 796.39.

BF-DPN Yellow solid, reaction yield=100% (2.45 gr), sublimation yield=70%. ¹HNMR (500 MHz, 295K, C₂D₄Cl₂) δ 7.85 (d, J=7.4 Hz, 2H), 7.62 (d, J=8.1 Hz, 2H), 7.51 (d, J=8.7 Hz, 2H), 7.41 (d, J=6.7 Hz, 4H), 7.31-7.23 (br, 12H), 7.15 (d, J=7.9 Hz, 4H), 7.05-7.02 (br, 4H), 1.36 (s, 12H). ¹³CNMR (125 MHz, 295K, C₂D₄Cl₂) δ 155.41, 153.85, 148.21, 147.51, 144.80, 140.07, 139.12, 134.40, 131.20, 129.63, 128.10, 127.29, 126.81, 125.12, 124.29, 123.68, 123.01, 122.80, 120.91, 120.39, 119.64, 119.02, 46.98, 27.13. HRMS (MALDI+) calculated m/z for [C₅₀H₄₀N₂+H]+ is 694.33, found 694.27.

BF-DPA Yellow solid, reaction yield=70% (0.765 gr), sublimation yield=69%. ¹HNMR (500 MHz, 295K, C₂D₄Cl₂) δ 8.04 (s br, 2H), 7.78 (d, 2H), 7.65 (d, 2H), 7.63 (d, J=8.0 Hz, 2H), 7.51 (d, 2H), 7.41 (d, J=6.9 Hz, 2H), 7.32-7.26 (s br, 12H), 7.20 (d, J=7.9 Hz, 4H), 7.10-7.07 (br, 4H), 1.40 (s, 12H). *carbon spectra could not be obtained due to solubility. HRMS (MALDI+) calculated m/z for [C₅₆H₄₄N₂+H]+ is 744.35, found 744.13.

### Density Functional Theory

All quantum chemical calculations were performed using Jaguar, version 2020-4, Schrodinger, Inc., New York, NY, 2021. (See A. D. Bochevarov, E. Harder, T. F. Hughes, J. R. Greenwood, D. A. Braden, D. M. Philipp, D. Rinaldo, M. D. Halls, J. Zhang, R. A. Friesner, “Jaguar: A High Performance Quantum Chemistry Software Program with Strengths in Life and Materials Sciences”, Int. J. Quantum Chem., 2013, 113, 2110-2142). All geometries were optimized using the B3LYP-D3 functional and the 6-31G⁺⁺** basis set. The Supporting Information contains the xyz coordinates for each of the five donor absorbers and B4PymPm, the simulated absorption spectra, the fifteen lowest energy roots determined by TD-DFT, and molecular orbital visualizations.

### Materials

All starting reagents for the syntheses of the donor absorbers were purchased from Sigma Aldrich and TCI and were used as received. Anhydrous toluene was purchased from Sigma Aldrich and used as received. The detailed synthetic procedures and characterizations for BF-DPP, BF-DPN, BF-DPA, and BF-DPT are found in the Supporting Information. B4PymPM, BF-DPB, TPBi, and BPhen were purchased from Luminescence Technology Corp. and used as received. The evaporation metals Li and Al, as well as MoO₃ were purchased from Sigma Aldrich, and were used as received.

### Materials Characterization

Both proton nuclear magnetic resonance (¹HNMR) spectra and carbon nuclear magnetic resonance (¹³CNMR) spectra were recorded on a Bruker 500 AVANCE equipped with a cryoprobe (500 and 125 MHz, respectively) at 295 K for BF-DPN, BF-DPA, and BF-DPT. Chemical shifts for protons are reported in parts per million (ppm) referenced to residual protium in the NMR solvent (C₂D₄Cl₂=3.72 ppm or DMSO=2.5 for BF-DPP). Chemical shifts for carbon are reported in parts per million downfield and are referenced to the carbon resonance of the solvent peak (C₂D₄Cl₂=43.6 ppm or DMSO=39.52 for BF-DPP). NMR splitting patterns are as follows: s=singlet, d=doublet, t=triplet, q=quartet, m=multiplet, br=broad. All coupling constants (J) are in hertz (Hz). High-resolution mass spectra were collected by matrix-assisted laser desorption/ionization (MALDI), and dithranol was used as the matrix.

Thermal transitions were measured on a PerkinElmer DSC-8500 differential scanning calorimeter. The scan rate was 10° C./min unless otherwise noted. Thermogravimetric analysis was performed on a Perkin Elmer TGA-8000 equipped with gas chromatography (GC) and mass spectrometry capabilities. The decomposition temperature was taken to be the temperature at which 5% weight loss was observed while heating at 10° C./min in nitrogen. The donor absorbers have T_{d}'s between 377° C.-397° C. Table 1 contains the T_{d} and Tₘ for each compound.

### Thin-Film Deposition and Device Fabrication

All substrates were cleaned prior to film deposition using sequential sonication in deionized water, acetone, and isopropyl alcohol, followed by rapid drying in a stream of N₂ gas. Pre-patterned ITO-coated substrates for device fabrication were purchased commercially from Luminescence Technology Corp. and treated with UV-ozone for 10 minutes following solvent cleaning. All organic layers, metals (Li and Al), and MoO₃ were deposited in an Angstrom Engineering thermal evaporator at chamber base pressures<5×10⁻⁷ Torr and growth rates between 0.1 Å/s and 2.5 Δ/s. The thicknesses of the films were monitored during deposition using a quartz crystal microbalance; thicknesses were separately calibrated or directly measured with a Woollam M-2000 spectroscopic ellipsometer, or a Bruker NanoMan atomic force microscope in the case of metal layers. Solar cell active areas of 9 mm² were defined as the overlap between a 3 mm wide strip of ITO on the pre-patterned substrates, and a 3 mm wide top electrode (either Al or ITO) defined with a shadow mask.

Top ITO electrodes for transparent devices were deposited in an Angstrom Engineering dielectric sputtering chamber via radio frequency sputtering of a 3″ diameter In₂O₃/SnO₂ (90/10 wt. %) sputtering target at 45 W to produce a growth rate of 0.22 Å/s. The sputtering gas was pure Ar flowing at 15 sccm, and the pumping rate was set to produce a process pressure of 3 mTorr during sputtering. The substrate was placed as far away from the target as possible during sputtering (about 25 cm), and the growth rate was monitored with a quartz crystal microbalance after thickness calibration with an α-step profilometer. A sheet resistance of 128±3Ω/□ was measured for 40-nm thick ITO films using a four-point probe measurement setup. After depositing ITO top electrodes, most OPVs were either partially shunted or fully short-circuited. A 5 s to 30 s reverse bias treatment at −10V to −20V was used to remove these shunts and recover diodic J-V characteristics.

### Photovoltaic Measurements

Following device fabrication, EQE spectra were obtained for all solar cells. First, chopped (200 hz) monochromatic light was coupled into a broad-band optical fiber, and the solar cells were illuminated by the optical fiber in an “underfilled” configuration. The photocurrent response of a Si reference cell with a known spectral responsivity was first measured as a function of wavelength with a Stanford Instruments SR810 lock-in current amplifier to calibrate the photon flux incident at each wavelength. Measuring the wavelength-dependent photocurrent produced by the NUV-OPVs in response to this illumination and dividing it by the calibrated photon flux yields EQE. To calculate the J_{SC} the NUV-OPVs would produce under AM1.5G solar illumination, the product of the AM1.5G solar flux and EQE spectra were integrated across all wavelengths. The results of these integrations are tabulated in Table 2 as J_{SC,EQE}. Additionally, J-V characteristics of all solar cells in this work were measured with a Keithley 2365 source measurement unit under illumination from a Xe-arc lamp to evaluate their photovoltaic performance. Due to the large spectral mismatch between the NUV-OPVs and standard photovoltaic reference cells, such as Si, the initial intensity of the solar simulator was tuned to produce a J_{SC,J-V} equivalent to the calculated J_{SC,EQE} from the EQE measurement on a conventional OPV with BF-DPB. As a result, the photocurrent from EQE and J-V measurements closely match for BF-DPB solar cells and others with a similar spectral response range as shown in Table 2, but deviate by several percent for the BF-DPN and BF-DPA cells, which absorb more visible photons.

### Transmittance Spectra Measurements and Absorption Coefficient Determination

Transmittance spectra were collected on an Agilent Technologies UV-Vis Cary 5000 spectrometer. Transmittance spectra of 20-nm thick films of each of the five donor absorbers and B4PymPm were collected on quartz and background corrected with a blank quartz slide. Absorption coefficients were calculated from background-corrected transmittance spectra following:

α=x ln(1−% T) (1)

where x is the film thickness, in cm.

Transmittance spectra on full-stack OPVs were measured through an iris with a 1-mm diameter. An empty sample beam was used as the background spectrum, such that the transmittance spectra incorporate the absorption/reflection of all the layers in the device stack including the glass substrate.

### Ultraviolet and Inverse Photoelectron Spectroscopy

Thin films of 7-20 nm were measured using ultraviolet photoelectron spectroscopy (UPS) and inverse photoelectron spectroscopy (IPES) in an ultrahigh vacuum (UHV) chamber with a base pressure of 5×10⁻¹⁰ Torr. Each sample was transferred from a N₂ glovebox to UHV without air exposure. Occupied states were measured using UPS He I photon line of a helium discharge lamp, where hv=21.22 eV. Unoccupied states were measured using IPES in isochromat mode, setup described elsewhere. The energy resolution for UPS and IPES is 150 meV and 450 meV, respectively.

### Photometric Calculations

Average photopic-response-weighted transmittance values were calculated from the transmittance spectra shown in FIG. 3f as follows:

$$A P T = \frac{\int T ( λ ) V ( λ ) \partial λ}{\int V ( λ ) \partial λ} \qquad ( 2 )$$

where T(λ) is the transmittance of the OPV, V(λ) is the photopic luminosity function, and the integration bounds encompass all visible wavelengths from 380 nm to 740 nm. For further photometric calculations (CRI, CCT, and CIE [x,y] chromaticity coordinates), an effective spectral power distribution for each transmittance spectrum was generated by multiplying the AM1.5G spectrum by the transmittance at each wavelength. The CRI, CCT, and CIE [x,y]chromaticity coordinates were then calculated for these effective illuminants. Color rendering indexes were calculated using the method outlined by R. Lunt (2012).^{[13]} We employed AM1.5G as the reference spectrum for these calculations rather than the CIE D65 standard illuminant for better applicability to window applications (i.e. the CRI of AM1.5G illumination is defined as 100). Tristimulus values were calculated by integrating the product of the spectral power distribution of each NUV—OPV and the three CIE color matching functions. The tristimulus values were then mapped onto the CIE 1931 color space to identify their CIE (x,y) coordinates via x=X/(X+Y+Z) and y=Y/(X+Y+Z). The CCT of each OPV was calculated by mapping its tristimulus values onto the CIE 1960 uniform chromaticity space via u=4X/(X+15Y+3Z) and v=6Y/(X+15Y+3z) and identifying the black body temperature of the closest point on the Planckian locus.

1. A compound of Formula (I): 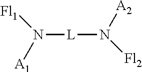
wherein A₁ and A₂ are independently selected from the group consisting of aryl and heteroaryl; and
wherein Fl₁ and Fl₂ are independently selected form the group consisting of fluorenyl and substituted fluorenyl; and
wherein L is selected from the group consisting of a fused aromatic ring structure and oligoarylene, wherein the oligoarylene comprises at least three arylene or heteroarylene units.

2. The compound of claim 1, wherein L is the fused aromatic ring structure.

3. The compound of claim 2, wherein the fused aromatic ring structure is naphthalene.

4. The compound of claim 2, wherein the fused aromatic ring structure is an acene.

5. The compound of claim 2, wherein fused aromatic ring structure comprises one or more heteroarylene units.

6. The compound of claim 5, wherein the heteroarylene units comprise thiophene, pyrrole, azole, or combinations thereof.

7. The compound of claim 1, wherein A₁ and A₂ are each aryl.

8. The compound of claim 1, wherein at least one or Fl₁ and Fl₂ is substituted with one or more alkyl or alkenyl substituents.

9. The compound of claim 1, wherein at least one of Fl₁ and Fl₂ is of the formula: 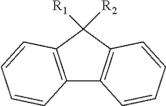
wherein R₁ and R₂ are independently selected from the group consisting of alkyl and alkenyl.

10. The compound of claim 1 having a peak absorption less than 440 nm.

11. The compound of claim 1 having a difference between the highest occupied molecular orbital (HOMO) and the lowest unoccupied molecular orbital (LUMO) of at least 2.5 eV.

12. The compound of claim 11, wherein the HOMO-LUMO difference is at least 2.8 eV.

13. An organic photovoltaic device comprising:
an anode;
a cathode; and
at least one active layer residing between the anode and the cathode, the active layer comprising an organic electron donor and organic electron acceptor, the organic electron donor comprising a compound of Formula (I): 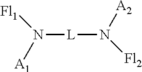
wherein A₁ and A₂ are independently selected from the group consisting of aryl and heteroaryl; and
wherein Fl₁ and Fl₂ are independently selected form the group consisting of fluorenyl and substituted fluorenyl; and
wherein L is selected from the group consisting of a fused aromatic ring structure and oligoarylene, wherein the oligoarylene comprises at least three arylene or heteroarylene units.

14. The organic photovoltaic device of 13, wherein L is the fused aromatic ring structure.

15. The organic photovoltaic device of 14, wherein the fused aromatic ring structure is naphthalene.

16. The organic photovoltaic device of claim 14, wherein the fused aromatic ring structure is an acene.

17. The organic photovoltaic device of claim 14, wherein fused aromatic structure comprises one or more heteroarylene units.

18. The organic photovoltaic device of claim 13, wherein the compound of Formula (I) has a difference between the highest occupied molecular orbital (HOMO) and the lowest unoccupied molecular orbital (LUMO) of at least 2.5 eV.

19. The organic photovoltaic device of claim 13, wherein peak absorbance of electromagnetic radiation by the active layer is in the range of 250 nm to 440 nm.

20. The organic photovoltaic device of claim 13, wherein the device is a single junction device.

21. The organic photovoltaic device of claim 13, wherein the organic electron donor and organic electron acceptor for a bulk heterojunction architecture in the active layer.

22. The organic photovoltaic device of claim 13, wherein the active layer has a thickness of 50-300 nm.

23. The organic photovoltaic device of claim 13, wherein the device exhibits an inverted architecture.

24. The organic photovoltaic device of claim 23 further comprising an organic light outcoupling layer over the anode.

25. The organic photovoltaic device of claim 13 having an open circuit voltage (V_{oc}) of at least 1.5 V.

26. The organic photovoltaic device of claim 13 having an open circuit voltage (V_{oc}) of 1.5 V to 3 V.

27. The organic photovoltaic device of claim 13 having an average photopic-response-weighted visible transmittance of at least 75%.

28. The organic photovoltaic device of claim 13 having an average photopic-response-weighted visible transmittance of at least 80%.

29. The photovoltaic device of claim 13 having a color rendering index of at least 90.0.

30. The photovoltaic device of claim 13 having a color rendering index of at least 95.0.

31-42. (canceled)

43. A method comprising:
tuning UV-absorption of an organic electron donor via varying length of an oligoarylene or fused aromatic ring structure bridging two diamine moieties.

44. The method of claim 43, wherein the fused aromatic aromatic ring structure is an acene.

45. The method of claim 43, wherein the length is varied to provide the organic electron donor a difference between the highest occupied molecular orbital (HOMO) and the lowest unoccupied molecular orbital (LUMO) of at least 2.8 eV.

46. The method of claim 43, wherein the organic electron donor is a compound of Formula (I): 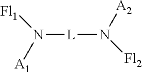
wherein A₁ and A₂ are independently selected from the group consisting of aryl and heteroaryl; and
wherein Fl₁ and Fl₂ are independently selected form the group consisting of fluorenyl and substituted fluorenyl; and
wherein L is selected from the group consisting of the fused aromatic ring structure and the oligoarylene.
